(19) United States
(12) Patent Application Publication
(10) Pub. No.: US 2022/0013207 A1
(43) Pub. Date: Jan. 13, 2022

(54) MEDICATION DISPENSER AND METHOD OF DISPENSING MEDICATION

(71) Applicant: Robert Kellett, Greenwood, SC (US)

(72) Inventor: Robert Kellett, Greenwood, SC (US)

(21) Appl. No.: 16/922,603

(22) Filed: Jul. 7, 2020

(51) Int. Cl. G16H 20/13; A61J 7/00; G16H 40/67; G16H 40/63

(52) U.S. Cl. CPC G16H 20/13; A61J 7/0084; A61J 2205/60; G16H 40/67; G16H 40/63; A61J 7/0069

(57) ABSTRACT

Medication dispensers and methods of dispensing medication are provided. A medication dispenser includes an exterior body defining an interior, and a distribution disk disposed within the interior, the distribution disk defining a plurality of compartments, the plurality of compartments disposed in at least one annular array, the distribution disk rotatably indexable about a central axis. The medication dispenser further includes a dispenser drawer in selective communication with one of the plurality of compartments disposed in the at least one annular array. The medication dispenser further includes a motor connected to the distribution disk, the motor operable to rotatably index the distribution disk about the central axis. Rotatable indexing of the distribution disk causes an adjustment in the one of the plurality of compartments disposed in the at least one annular array which is in communication with the dispenser drawer.

### FIELD

The present disclosure relates generally to medication dispensers and methods of dispensing medication.

### BACKGROUND

Medication dispensers are generally known, and are utilized by patients and medical personnel to organize and dispense required medications. In general, known medication dispensers include a plurality of compartments which may be labeled in accordance with a desired consumption schedule such as, for example, days of the week. A patient or medical personnel can load one or more medications into the dispenser and can divide the medications into the compartments as required for, for example, a particular prescription. This in turn allows the patient to take the medication as required in accordance with the consumption schedule by only accessing one compartment of medication at a time.

However, issues exist with known medication dispensers. For example, while known medication dispensers facilitate initial organization of the medication, there are no mechanisms in place for limiting the medication consumption to the consumption schedule. Accordingly, there are no safeguards in place to prevent, for example, a patient to develop a medication addiction, access more than one compartment at a time, and consume medication at a rate higher than the established consumption schedule. Conversely, there are also no mechanisms in place to encourage a patient to consume medication in accordance with the established consumption schedule.

Additionally, there are no mechanisms in place for reporting consumption data to, for example, medical personnel, legal personnel, or other responsible parties to confirm than medication is being consumed in accordance with an established consumption schedule.

Accordingly, improved medication dispensers and methods for dispensing medication are desired in the art. For example, medication dispensers and methods which can regulate the distribution and consumption of medication in accordance with an established consumption schedule, and/or which can facilitate reporting of consumption data, would be advantageous.

### BRIEF DESCRIPTION

Aspects and advantages of the medication dispensers and methods in accordance with the present disclosure will be set forth in part in the following description, or may be obvious from the description, or may be learned through practice of the technology.

In accordance with one embodiment, a medication dispenser is provided. The medication dispenser includes an exterior body defining an interior, and a distribution disk disposed within the interior, the distribution disk defining a plurality of compartments, the plurality of compartments disposed in at least one annular array, the distribution disk rotatably indexable about a central axis. The medication dispenser further includes a dispenser drawer in selective communication with one of the plurality of compartments disposed in the at least one annular array. The medication dispenser further includes a motor connected to the distribution disk, the motor operable to rotatably index the distribution disk about the central axis. Rotatable indexing of the distribution disk causes an adjustment in the one of the plurality of compartments disposed in the at least one annular array which is in communication with the dispenser drawer.

In some embodiments, the medication dispenser further includes a door movable to selectively provide access to the dispenser drawer from one or more compartments in a linear array which is in communication with the dispenser drawer. In some embodiments, the medication dispenser further includes an auxiliary motor connected to the door, the auxiliary motor operable to move the door.

In some embodiments, the medication dispenser further includes a mobile computing device in operative communication with the motor and/or the auxiliary motor, the mobile computing device including a processor and a user interface. The mobile computing device controls operation of the motor and/or the auxiliary motor based on a user input to the mobile computing device.

In accordance with another embodiment, a computer-implemented method of dispensing medication is provided. The computer-implemented method includes receiving, by a computing system comprising a mobile computing device, at least one user input. The computer-implemented method further includes correlating, by the computing system, the at least one user input with stored information, wherein the stored information includes date and/or time information. The computer-implemented method further includes selectively generating, by the computing system, a command to operate a motor of a medication dispenser based on the correlation of the at least one user input and the stored information. Generation of the command causes indexing of a distribution disk which causes adjustment of a compartment of a plurality of compartments defined by the distribution disk which is in communication with a dispenser drawer of the medication dispenser. The computer-implemented method further includes storing, by the computing system, the at least one user input, the stored information, and a confirmation of the selective generation.

In some embodiments, the computer-implemented method further includes selectively generating, by the computing system, a command to operate an auxiliary motor of the medication dispenser based on the correlation of the at least one user input and the stored information, wherein generation of the command to operate the auxiliary motor causes movement of a door to selectively provide access to the dispenser drawer from one or more compartments in a linear array which is in communication with the dispenser drawer.

In some embodiments, the computing system includes the mobile computing device and a computing system, wherein the receiving step is performed by the mobile computing device, and wherein the storing step is performed by the computing system.

In accordance with another embodiment, one or more non-transitory computer-readable media that store instructions that, when executed by one or more processors, cause the one or more processors to perform operations, the operations including one or more methods steps as discussed herein, are provided.

These and other features, aspects and advantages of the present medication dispensers and methods will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present medication dispensers and methods, including the best mode of making and using the present systems and methods, directed to one of ordinary skill in the art, is set forth in the specification, which makes reference to the appended figures, in which:

FIG. 1 is perspective view of a medication dispenser in accordance with embodiments of the present disclosure;

FIG. 2 is a top perspective view of components of a medication dispenser, include a distribution disk and a dispenser drawer, in accordance with embodiments of the present disclosure;

FIG. 3 is a bottom perspective view of components of a medication dispenser, including a distribution disk, door, motor, and auxiliary motor, with the door in a closed position, in accordance with embodiments of the present disclosure;

FIG. 4 is a bottom perspective view of components of a medication dispenser, including a distribution disk, door, motor, and auxiliary motor, with the door in an intermediate open position, in accordance with embodiments of the present disclosure;

FIG. 5 is a bottom perspective view of components of a medication dispenser, including a distribution disk, door, motor, and auxiliary motor, with the door in a fully open position, in accordance with embodiments of the present disclosure;

FIG. 6 is a perspective view of a motor assembly which includes a motor in accordance with embodiments of the present disclosure;

FIG. 7 illustrates an example computing environment in accordance with embodiments of the present disclosure;

FIG. 8 illustrates a block diagram of a method of dispensing medication in accordance with embodiments of the present disclosure; and

FIG. 9 illustrates a block diagram of a computing system in accordance with embodiments of the present disclosure.

### DETAILED DESCRIPTION

Reference now will be made in detail to embodiments of the present medication dispensers and methods, one or more examples of which are illustrated in the drawings. Each example is provided by way of explanation, rather than limitation of, the technology. In fact, it will be apparent to those skilled in the art that modifications and variations can be made in the present technology without departing from the scope or spirit of the claimed technology. For instance, features illustrated or described as part of one embodiment can be used with another embodiment to yield a still further embodiment. Thus, it is intended that the present disclosure covers such modifications and variations as come within the scope of the appended claims and their equivalents.

The detailed description uses numerical and letter designations to refer to features in the drawings. Like or similar designations in the drawings and description have been used to refer to like or similar parts of the invention. As used herein, the terms “first”, “second”, and “third” may be used interchangeably to distinguish one component from another and are not intended to signify location or importance of the individual components.

As used herein, the terms “upstream” (or “forward”) and “downstream” (or “aft”) refer to the relative direction with respect to fluid flow in a fluid pathway. For example, “upstream” refers to the direction from which the fluid flows, and “downstream” refers to the direction to which the fluid flows. The term “radially” refers to the relative direction that is substantially perpendicular to an axial centerline of a particular component, the term “axially” refers to the relative direction that is substantially parallel and/or coaxially aligned to an axial centerline of a particular component and the term “circumferentially” refers to the relative direction that extends around the axial centerline of a particular component. terms of approximation, such as “generally,” or “about” include values within ten percent greater or less than the stated value. When used in the context of an angle or direction, such terms include within ten degrees greater or less than the stated angle or direction. For example, “generally vertical” includes directions within ten degrees of vertical in any direction, e.g., clockwise or counter-clockwise.

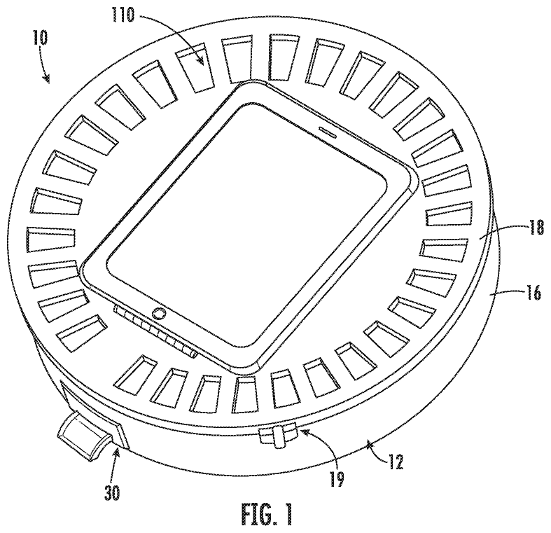

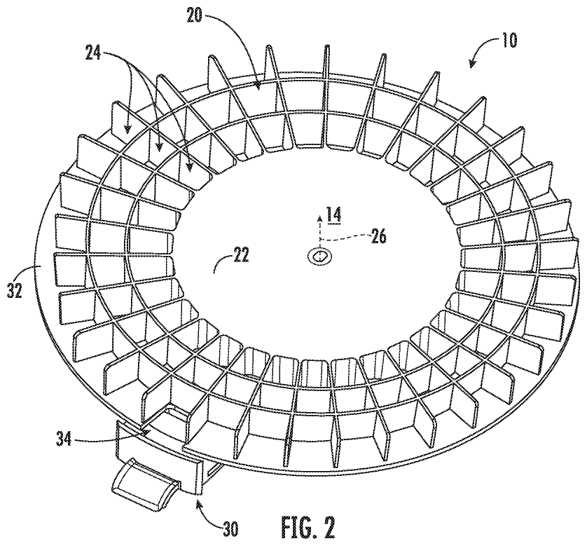

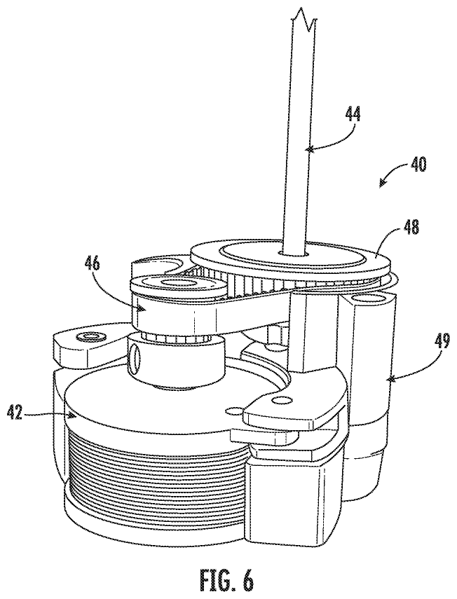

Referring now to FIGS. 1 through 9, the present disclosure is directed to improved medication dispenser and methods of dispensing medication. Such medication dispensers and methods advantageously include features to regulate the distribution and consumption of medication in accordance with established consumption schedules. Further such medication dispensers and methods can advantageously facilitate reporting of consumption data.

The advantages of medication dispensers and methods in accordance with the present disclosure are vast. For example, the present disclosure contemplates a medication dispenser that logs when a medication has been taken, and if it has not been taken provides the option to send push notifications to people who need to know, such as medical personnel, legal personnel, and/or caregivers. By logging this data, one has created a compliance index enabling medical personnel, legal personnel, and/or caregivers to make more informed decisions regarding medication management.

Examples of such advantages include: home health nurses can monitor the antibiotic compliance of patients at home with wound care needs; adult protective services can check to see if the elderly are getting the medications they are supposed to get from their caregivers, thus providing a proxy indicator of their care; the legal system can order drug rehabilitation programs for opioid addictions, and parole officers can report on those patient's compliance; child protective services can check on the parents of dependent children to make sure they are not taking their controlled substances recklessly but only as prescribed; if a DUI case is brought against a patient and they are accused of medication abuse the defense will have a third party documented answer to prove compliance, and the same goes for the surgeon who is taking pain medication; medication can't be described to a suicidal patient with built-in safeguard against overdosing; an insurance company can reward people who are compliant with taking medication on a required consumption schedule or, alternatively, can increase the rates of those who are not better controlling risks.

With reference now to FIGS. 1 through 6, embodiments of medication dispensers 10 in accordance with embodiments of the present disclosure are provided. A medication dispenser 10 includes an exterior body 12 which defines an interior 14. Exterior body 12 may include, for example, a base 16 and a top 18 which are connectable together to form exterior body 12 and define interior 14 therebetween.

In exemplary embodiments, the exterior body 12 may be lockable such that the interior 14 and components therein, such as a distribution disk 20 disposed within the interior 14, are inaccessible (except via, for example, a distribution drawer as discussed herein). For example, a suitable lock 19 may be provided, and may be utilized to lock the base 16 and top 18 together to prevent separation of the base 16 and top 18 from each other when locked. The lock 19 can be unlocked to provide such separation and/or access. In some embodiments, medical personnel, legal personnel, and/or caregivers may be able to unlock and lock the lock 19. This allows for such suitable personnel to load medication into the medication dispenser, input a required consumption schedule, etc. Further, in some embodiments, a patient may not be able to lock and unlock the lock 19, so that the patient cannot access medication therein except on a defined consumption schedule (as discussed herein).

A medication dispenser 10 may further include a distribution disk 20 disposed within the interior 14. Distribution disk 20 may include a body 22 which defines a plurality of compartments 24. In exemplary embodiments the distribution disk has an annular shape. Further, in exemplary embodiments, the plurality of compartments 24 are disposed in one or more annular arrays. For example, in the embodiment shown, the plurality of compartments 24 are disposed in three concentric annular arrays. Alternatively, the plurality of compartments 24 may be disposed in one, two, four or more annular arrays which may, for example, be concentric. Each annular array may include a defined number of compartments 24. For example, in the embodiment shown, thirty compartments 24 are provided in each annular array. Alternatively, more or less than thirty compartments may be provided in an annular array.

Further, in exemplary embodiments, the plurality of compartments 24 may be disposed in a plurality of linear arrays, which in exemplary embodiments may be radially extending linear arrays. For example, a linear array may include one compartment 24 in each of the plurality of annular arrays. Accordingly, each compartment 24 may be part of an annular array and part of a linear array. In the embodiment shown, the plurality of compartments 24 are disposed in thirty linear arrays. Alternatively, more or less than thirty linear arrays may be provided. Each linear array may include a defined number of compartments 24. For example, in the embodiment shown, three compartments 24 are provided in each linear array. Alternatively, more or less than three compartments may be provided in a linear array.

In exemplary embodiments, each compartment 24 may correspond with a date and/or time during which a medication should be consumed. For example, each compartment 24 in a linear array may correspond to a time during a specific day, such as (in the case of three compartments) morning, afternoon, and evening. These time periods are periods during which a medication provided in that compartment 24 should be consumed in accordance with a defined medication consumption schedule. Further, each compartment 24 in an annular array may correspond to a day, such as a day of a month, week, etc. These days are days during which a medication provided in that compartment 24 should be consumed in accordance with a defined medication consumption schedule.

Distribution disk 20 may be rotatable indexable about a central axis 26. Such rotatable indexing may, in exemplary embodiments, be through a plurality of positions at least equal to the number of compartments in an annular array of compartments 24. For example, in the embodiment wherein each annular array has 30 compartments, the distribution disk 20 may be rotatable indexable through at least 30 positions. Each position may correspond with one of the plurality of compartments 24 in the annular array.

Medication dispenser 10 may further include a dispenser drawer 30. Dispenser drawer 30 may facilitate access to medication that is distributed by medication dispenser 10 for consumption by a patient. For example, dispenser drawer 30 may be movable into and out of the interior 14, such as along a radial direction, by a patient. Medication that is dispensed by medication dispenser 10 may be dispensed into the dispenser drawer 30, such that the patient can access and consume the medication via the dispenser drawer 30.

Dispenser drawer 30 may be in selective communication with various of the compartments 24 in order to provide such access. For example, dispenser drawer 30 may be in selective communication with one of the plurality of compartments 24 in an annular array, such as one of the plurality of compartments 24 in each annular array, such as the plurality of compartments 24 in a linear array, when the distribution disk 20 is rotatably indexed to a particular indexed position. Indexing of the distribution disk 20 may cause rotation of the distribution disk such that dispenser drawer 30 may be in communication with a different one of the plurality of compartments 24 in an annular array, such as one of the plurality of compartments 24 in each annular array, such as plurality of compartments 24 in a linear array. Indexing of the distribution disk in a full rotational cycle may cause each one of the plurality of compartments 24 in an annular array to have been in communication with the dispenser drawer 30 at one indexed position during such cycle. In this manner, rotatable indexing of the distribution disk 20 causes an adjustment in the one or more compartments 24 disposed in the at least one annular array which is in communication with the dispenser drawer 30.

Medication dispenser 10 may further include, for example, a distribution plate 32 disposed between the distribution disk 20 and the dispenser drawer 30. Distribution disk 20 may be rotatable relative to the distribution plate 32. The distribution disk 20 may include one or more apertures 34 defined therein. When a compartment 24 is in communication with the dispenser drawer 30, such communication may be through an aperture 34.

To facilitate rotational indexing of the distribution disk 20, a motor 42 may be connected to the distribution disk 20. Operation of the motor 42 may rotatably index the distribution disk 20 about the central axis 26. In some embodiments, motor 42 may be directly connected to the distribution disk 20. In other embodiments, motor 42 may be indirectly connected to the distribution disk 20, such as via a shaft 44. For example, motor 42 may be a component of a motor assembly 40 which includes the motor 42 and shaft 44. Motor 42 may rotate shaft 44, which may extend between motor 42 and distribution disk 20 and thus rotate the distribution disk 20 due to operation of the motor 42. In some embodiments, motor assembly 40 may further include a belt 46 and a sprocket 48 which are connected between motor 42 and shaft 44. Operation of the motor 42 may cause rotation of the belt 46, which in turn may drive rotation of the sprocket 48, which in turn may drive rotation of the shaft 44. In exemplary embodiments, a casing 49 may secure the motor assembly 40 components together. Casing 49 may be formed from a suitably pliant material to provide suitable cushioning and shock absorption for the various motor assembly 40 components relative to each other and the medication dispenser 10 generally.

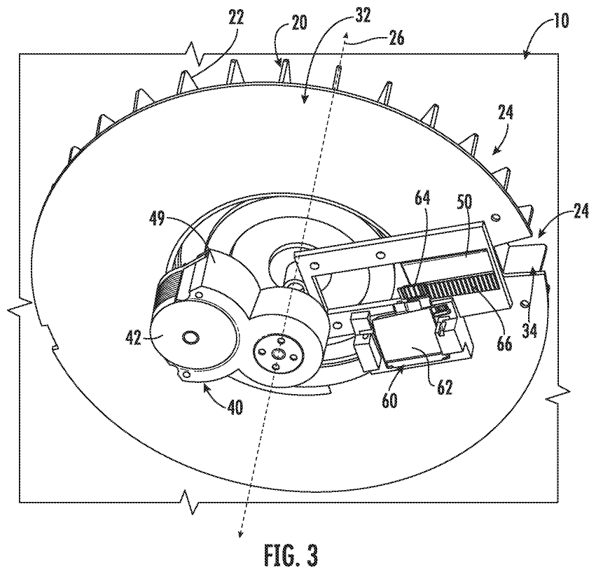

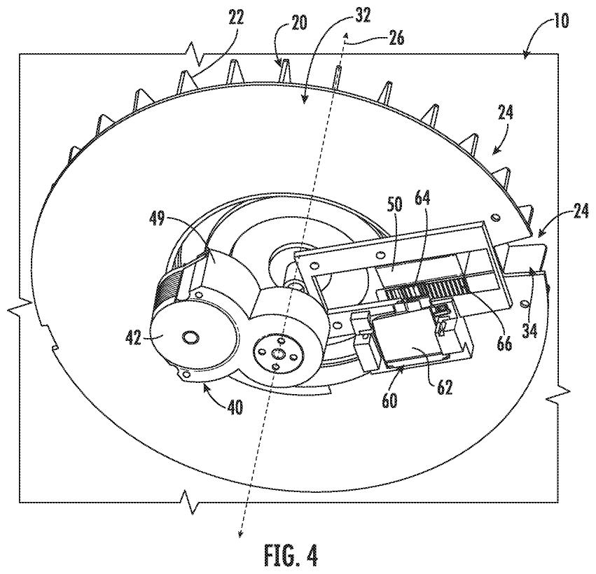

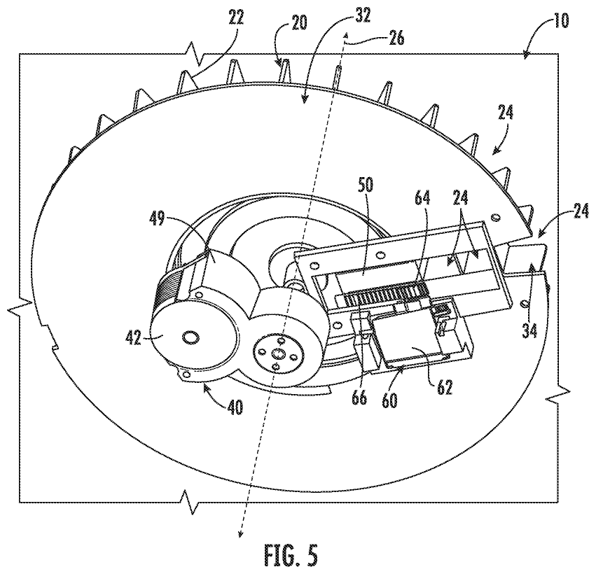

Medication dispenser 10 may further include a door 50 disposed in the interior 14. Door 50 may be movable, such as in exemplary embodiments radially movable, to selectively provide access to the dispenser drawer 30 from one or more of the compartments 24 in a linear array which is in communication with the dispenser drawer 30. For example, door 50 may be movable between various positions, such as a closed position as illustrated in FIG. 3, an intermediate open position as illustrated in FIG. 4, and a fully open position as illustrated in FIG. 5. In each position relative to the other positions, either a different number of compartments 24 in a linear array may be in communication with the dispenser drawer 30 or a different compartment 24 of a linear array may be in communication with the dispenser drawer 30. For example, in the closed position as illustrated in FIG. 3, only a single compartment 24 in a linear array is in communication with the dispenser drawer 30. In the intermediate open position as illustrated in FIG. 4, two compartments 24 in a linear array are in communication with the dispenser drawer 30. In the fully open position as illustrated in FIG. 5, three compartments in a linear array are in communication with the dispenser drawer 30.

In exemplary embodiments, door 50 may be movable between rotational indexing of the distribution disk 20. For example, when distribution disk 20 is in an indexed position rotationally, door 50 may move from a closed position to an intermediate open position, and then to a fully open position, at various intervals. After movement to the fully open position, the door 50 may be moved to the closed position and the distribution disk 20 rotationally indexed, and the door movement may then be repeated.

To facilitate movement of the door 50, an auxiliary motor 62 may be connected to the door 50. Operation of the auxiliary motor 62 may move the door 50. In some embodiments, the auxiliary motor 62 may be directly connected to the door 50. In other embodiments, auxiliary motor 62 may be indirectly connected to the door 50. For example, in exemplary embodiments, auxiliary motor 62 may be a component of an auxiliary motor assembly 60 which includes the auxiliary motor 62, a pinion gear 64, and a rack 66. The rack 66 may be mounted to the door 50. Rotation of the auxiliary motor 62 may cause rotation of the pinion gear 64, which may in turn cause movement of the rack 66, thus causing movement of the door 50.

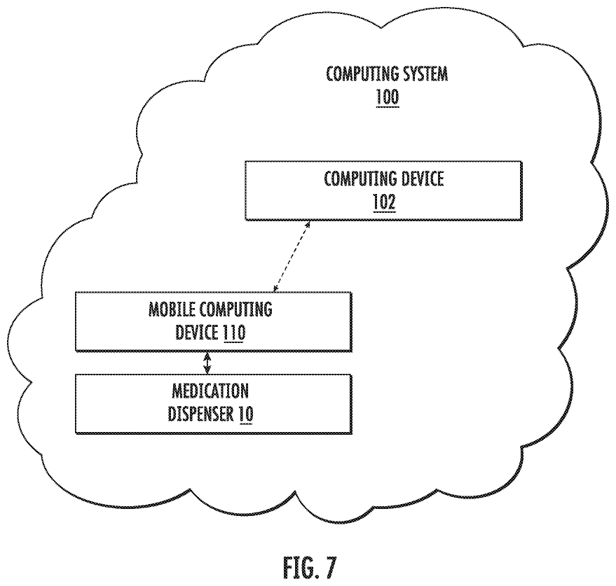

With reference now also to FIGS. 7 through 9, medication dispensers 10 in accordance with the present disclosure may further include computing systems 100 and/or mobile computing devices 110 thereof which are in communication with and facilitate operation of the medication dispensers 10 and components thereof, such as the motor 42 and auxiliary motor 62, as discussed herein. A mobile computing device 110 may, for example, be connected to the exterior body 12, such as hingedly connected as shown. Alternatively, mobile computing device 110 may be separate from but the exterior body 12 but in communication with the necessary components, such as the motor 42 and auxiliary motor 62. Motor 42 and auxiliary motor 62 (and other suitable components) may be directly wired to the mobile computing device 110 to receive and process instructions from the mobile computing device 110, or may themselves include controllers or other suitable computer systems in communication with the mobile computing device 110 to receive and process instructions from the mobile computing device 110.

Mobile computing device 110 may include one or more processors and one or more user interfaces, as discussed herein. Each user interface may facilitate the input of user inputs by, for example, a patient, in order to utilize the medication dispenser 10. Further, each user interface may facilitate the input of user inputs by, for example, medical personnel, legal personnel, or a caregiver to input, for example, a consumption schedule.

Computing system 100 enables the distribution of medication using medication dispensers 10 in accordance with an established consumption schedule which can, for example, be programmed into the computing system 100. Further, computing system 100 enables tracking and reporting of consumption data. Computing system 100 includes or is in communication with one or more mobile computing devices 110. Although the mobile computing device 110 is shown separate from computing system 100, it will be appreciated that mobile computing devices 110 in accordance with the present disclosure may be considered as part of the computing system 100. Computing system 100 may include one or more computing devices 102 in communication with each of the mobile computing devices 110. Computing system 100 can be implemented using one or more computing devices 102, such as one or more servers, client devices, and/or network devices. Various components of computing system 100 may be implemented in hardware, software, or a combination of hardware and software.

Mobile computing devices 110 and computing system 100 may include one or more network interfaces that are used to communicate with other computing devices over a network (not shown). The network interface can include any suitable components for interfacing with one more networks, including for example, transmitters, receivers, ports, controllers, antennas, or other suitable components. The network can be any type of communications network, such as a local area network (e.g. intranet), wide area network (e.g. Internet), cellular network, or some combination thereof. The network can also include a direct connection between a mobile computing device 110 and the computing system 100. In general, communication between the computing system 100 and a mobile computing device 110 and other suitable components in the medication dispenser such as motor 42 and/or auxiliary motor 62 can be carried via network interface using any type of wired and/or wireless connection, using a variety of communication protocols (e.g. TCP/IP, HTTP, SMTP, FTP), encodings or formats (e.g. HTML, XML), and/or protection schemes (e.g. VPN, secure HTTP, SSL). It is noted that while reference is made to mobile computing devices any client or other suitable computing device may be used to perform influence can and other operations as described herein.

As discussed, mobile computing device 110 may be in operative communication with the motor 42, and may further be in communication with the auxiliary motor 62. Mobile computing device 110 may control operation of the motor 42 and auxiliary motor 62 based on one or more user inputs, such as via one or more user interfaces, to the mobile computing device 110. For example, a user input may include identification data, which may for example, be input as a typed password, spoken word or signal, visual signal (e.g. facial recognition), etc., via a user interface. Such user inputs may be correlated with approved user inputs saved in the mobile computing device 110 and/or computing system 100 generally to identify whether an approved user, e.g. the patient, is trying to access medication in the medication dispenser 10.

The computing device 110 may further control operation of the motor based on stored information, either in the computing device 110 or computing system 100 generally. The stored information may include date and/or time information, such as current date and/or time information as well as date and/or time information in an established consumption schedule programmed into the computing device 110 or computing system 100 generally. In exemplary embodiments, for example, the computing device 110 may, upon correlation of a user input with an approved user input, further compare the current date and/or time with the date and/or time information in an established consumption schedule. If the current date and/or time matches the appropriate date and/or time information in the established consumption schedule, access to the scheduled medication in the medication dispenser 10 may be granted, and the motor 42 and/or auxiliary motor 62 may be moved accordingly. However, if the current date and/or time does not match the appropriate date and/or time information in the established consumption schedule, access to the scheduled medication in the medication dispenser 10 may not be granted, and the motor 42 and/or auxiliary motor 62 may not be moved accordingly.

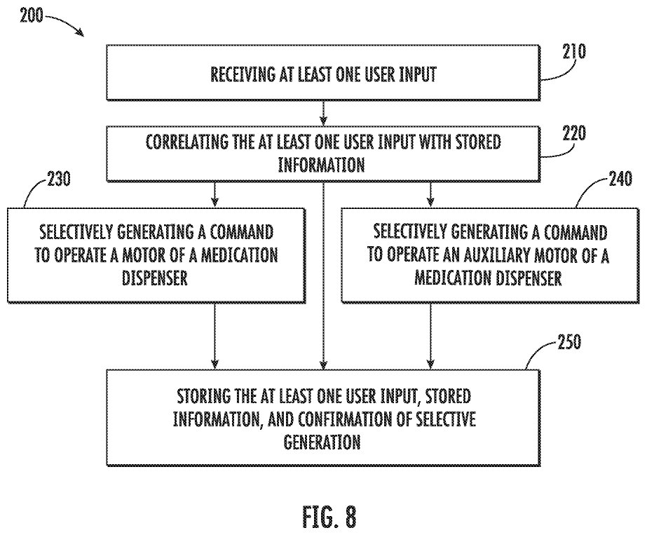

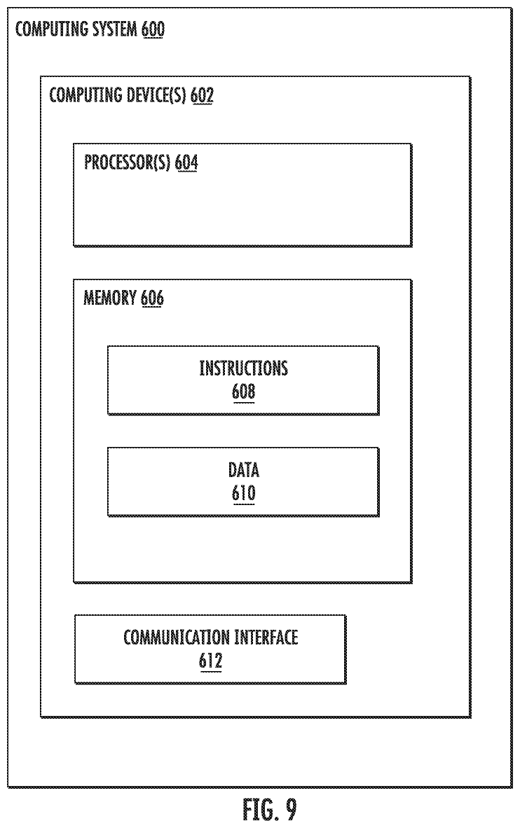

FIG. 8 is a flowchart illustrating one embodiment of a method 200 of dispensing medication in accordance with the present disclosure. One or more portions or steps of method 200 can be implemented by one or more computing devices such as, for example, a computing device 152 of computing system 150, a mobile computing device 160, and/or a computing system 600 as illustrated in FIG. 9. One or more portions or steps of the method 200 described herein can be implemented as an algorithm on the hardware components of the devices described herein to, for example, dispense medication, operate a medication dispenser 10, regulate the distribution and consumption of medication in accordance with an established consumption schedule, and/or track, record, and/or report consumption data. Although FIG. 8 depicts steps performed in a particular order for purposes of illustration and discussion, method 200 is not limited to the particularly illustrated order or arrangement. The various steps of the methods disclosed herein can be omitted, rearranged, combined, and/or adapted in various ways without deviating from the scope of the present disclosure.

Method 200 may include, for example, the step 210 of receiving at least one user input as discussed herein. As discussed, a patient may for example input the user input(s). A computing system 100, such as a mobile computing device 110 thereof, may receive the at least one user input.

Method 200 may further include, for example, the step 220 of correlating the at least one user input with stored information as discussed herein. For example, such correlation may include correlating the user input with authorized user inputs to determine whether the user inputting the user input is granted access to the medication dispenser 10. Further and/or if access is granted (because, for example, the user input matches an authorized user input stored in the computing system 100), real time information such as the current date and time may be compared with other stored information such as stored dates and/or times in a consumption schedule. If the real time information matches or fits within an approved window of stored information, access may be granted to the medication dispenser. A computing system 100 may provide such correlation.

Notably, if access is not granted at step 210 or step 220, no further steps may occur.

Method 200 may further include, for example, the step 230 of selectively generating a command to operate a motor 42 of the medication dispenser 10 based on the correlation of the at least one user input and the stored information. For example, such step 230 may occur if the user input(s) match stored authorized user inputs and specified real time information matches stored information, and may not occur if the user input(s) do not match stored authorized user inputs and/or specified real time information does not match stored information. Generation of the command may cause indexing of the distribution disk 20, which may in turn cause adjustment of the one or more compartments 24 which are in communication with a dispenser drawer 30 of the medication dispenser 10. A computing system 100 may provide such command generation.

Method 200 may further include, for example, the step 240 of selectively generating a command to operate an auxiliary motor 62 of the medication dispenser 10 based on the correlation of the at least one user input and the stored information. For example, such step 240 may occur if the user input(s) match stored authorized user inputs and specified real time information matches stored information, and may not occur if the user input(s) do not match stored authorized user inputs and/or specified real time information does not match stored information. Generation of the command may cause operation of the auxiliary motor 62, which in turn may cause movement of a door 50 to selectively provide access to the dispenser drawer 30 from one or more compartments 24 in a linear array which is in communication with the dispenser drawer 30. A computing system 100 may provide such command generation.

For example, each compartment 24 in medication dispenser 10 may be preloaded with a specified dose of one or more medications which is to be taken at a specified date and/or time on a consumption schedule. The consumption schedule (and dates/times thereof) may be stored in computing system 100. If a user input correlates with a stored authorized user input and the real time date and/or time for a dose of medication match as discussed above with respect to step 220, steps 230 and/or 240 may occur. For example, upon such indexing in accordance with step 230, a compartment 24 or plurality of compartments 24 (such as a linear array thereof) may become aligned with the dispenser drawer 30. At least one of such aligned compartments 24 may become in communication with the dispenser drawer 30, such that the medication therein is transmitted (e.g. via gravity) to the dispenser drawer 30. The user is then free to access this medication. Subsequently, if a user input correlates with a stored authorized user input and the subsequent real time date and/or time for a subsequent dose of medication match as discussed above with respect to step 220, steps 230 and/or 240 may occur. For example, upon such movement in accordance with step 240, another compartment 24 in a linear array of compartments may become in communication with the dispenser drawer 30, such that the medication therein is transmitted (e.g. via gravity) to the dispenser drawer 30. The user is then free to access this medication.

In some embodiments, step 240 may occur after steps 210 and 220 without step 230 occurring therebetween. In some embodiments, step 240 may occur along with (e.g. just before or simultaneously to) step 230 after steps 210 and 220.

A method 200 may further include, for example, the step 250 of storing the at least one user input, the stored information, and a confirmation of the selective generation at step 230 and/or 240. For example, a date and time stamp of an attempted access by a user due to a user input, as well as a confirmation of whether access was granted, may be stored. Such stored data may further be transmitted to authorized third parties as desired or required for a particular application.

FIG. 9 depicts a block diagram of an example computing system 600 that can be used by a control system 100, mobile computing device 110, controller, or other systems to implement methods and systems according to example embodiments of the present disclosure. As shown, the computing system 600 can include one or more computing device(s) 602. The one or more computing device(s) 602 can include one or more processor(s) 604 and one or more memory device(s) 606. The one or more processor(s) 604 can include any suitable processing device, such as a microprocessor, microcontroller, integrated circuit, logic device, or other suitable processing device. The one or more memory device(s) 606 can include one or more computer-readable media, including, but not limited to, non-transitory computer-readable media, RAM, ROM, hard drives, flash drives, or other memory devices.

The one or more memory device(s) 606 can store information accessible by the one or more processor(s) 604, including computer-readable instructions 608 that can be executed by the one or more processor(s) 604. The instructions 608 can be any set of instructions that when executed by the one or more processor(s) 604, cause the one or more processor(s) 604 to perform operations. The instructions 608 can be software written in any suitable programming language or can be implemented in hardware. In some embodiments, the instructions 608 can be executed by the one or more processor(s) 604 to cause the one or more processor(s) 604 to perform operations, such as the operations for generating performing implement and other scans to determine tracking indicia in accordance with processing stages of processing cycle utilizing a plurality of cutting implements, generate state data and association data associated with cutting implements, detect missing cutting implements, and initiate control actions associated with missing control elements as described above, and/or any other operations or functions of the one or more computing device(s) 602.

The memory device(s) 606 can further store data 610 that can be accessed by the processors 604. For example, the data 610 can include state data, association data, processing cycle and/or stages data, and user interface data, etc., as described herein. The data 610 can include one or more table(s), function(s), algorithm(s), model(s), equation(s), etc. according to example embodiments of the present disclosure.

The one or more computing device(s) 602 can also include one or more communication interfaces 612 (which may include user interfaces) used to communicate, for example, with the other components of system or a user. The communication interface 612 can include any suitable components for interfacing with one or more network(s), including for example, transmitters, receivers, ports, controllers, antennas, or other suitable components.

The technology discussed herein makes reference to computer-based systems and actions taken by and information sent to and from computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. A medication dispenser, comprising:
an exterior body including a base and a top and defining an interior, the top being separably connectable to the base;
a distribution disk disposed within the interior, the distribution disk defining a plurality of compartments, the plurality of compartments disposed in at least one annular array, the distribution disk rotatably indexable about a central axis;
a dispenser drawer in selective communication with one of the plurality of compartments disposed in the at least one annular array;
a motor connected to the distribution disk, the motor operable to rotatably index the distribution disk about the central axis, wherein rotatable indexing of the distribution disk causes an adjustment in the one of the plurality of compartments disposed in the at least one annular array which is in communication with the dispenser drawer;
a door movable to selectively provide access to the dispenser drawer from one or more compartments in a linear array which is in communication with the dispenser drawer; and
an auxiliary motor assembly including an auxiliary motor, a pinion gear, and a rack mounted to the door, wherein rotation of the pinion gear causes movement of the rack and the door.

2. The medication dispenser of claim 1, further comprising:
a mobile computing device in operative communication with the motor, the mobile computing device including a processor and a user interface, wherein the mobile computing device controls operation of the motor based on a user input to the mobile computing device.

3. The medication dispenser of claim 2, wherein the user input comprises identification data.

4. The medication dispenser of claim 2, wherein the mobile computing device further controls operation of the motor based on stored information, and wherein the stored information includes date and/or time information.

5. The medication dispenser of claim 2, wherein the mobile computing device is in communication with a computing system.

6. The medication dispenser of claim 1, wherein the plurality of compartments are disposed in a plurality of annular arrays such that the plurality of compartments is further disposed in a plurality of radially extending linear arrays, and wherein the dispenser drawer in selective communication with a linear array of compartments.

7. (canceled)

8. The medication dispenser of claim 1, wherein the door is radially movable.

9. (canceled)

10. The medication dispenser of claim 8, further comprising:
a mobile computing device in operative communication with the motor and the auxiliary motor, the mobile computing device including a processor and a user interface, wherein the mobile computing device controls operation of the motor and the auxiliary motor based on a user input to the mobile computing device, wherein the mobile computing device is in communication with a computing system.

11. The medication dispenser of claim 1, wherein separating the top from the base provides access to the entire distribution disk.

12. The medication dispenser of claim 1, wherein the exterior body is lockable such that the distribution disk is inaccessible.

13. A computer-implemented method of dispensing medication, comprising:
receiving, by a computing system comprising a mobile computing device, at least one user input, the user input comprising identification data;
correlating, by the computing system, the at least one user input with stored identification information;
correlating, by the computing system, real time date and time information with stored date and time information in a predetermined consumption window;
when the received user input corresponds with the stored identification information and when the real time date and time information falls within the predetermined consumption window the method further includes selectively generating, by the computing system, a command to operate a motor of a medication dispenser based on the correlation of the at least one user input and the stored information, the medication dispenser comprising an exterior body that includes a base and a top, wherein the top is separably connectable to the base, wherein generation of the command causes indexing of a distribution disk which causes adjustment of a compartment of a plurality of compartments defined by the distribution disk which is in communication with a dispenser drawer of the medication dispenser; and
storing, by the computing system, the at least one user input, the stored information, and a confirmation of the selective generation.

14. (canceled)

15. The computer-implemented method of claim 13, wherein the indexing is rotatable indexing.

16. The computer-implemented method of claim 13, further comprising selectively generating, by the computing system, a command to operate an auxiliary motor of the medication dispenser based on the correlation of the at least one user input and the stored information, wherein generation of the command to operate the auxiliary motor causes movement of a door to selectively provide access to the dispenser drawer from one or more compartments in a linear array which is in communication with the dispenser drawer.

17. The computer-implemented method of claim 13, wherein the movement of the door is linear movement.

18. The computer-implemented method of claim 13, wherein the computing system comprises the mobile computing device and a computing system, wherein the receiving step is performed by the mobile computing device, wherein the storing step is performed by the computing system, and wherein the mobile computing device is in wireless communication with the computing system.

19. The computer-implemented method of claim 13, further comprising:
separating the top from the base to provide access to the entire distribution disk.

20. The computer-implemented method of claim 13, wherein the mobile computing device is in wireless communication with the motor.
